(19) United States
(12) Patent Application Publication
(10) Pub. No.: US 2007/0156208 A1
(43) Pub. Date: Jul. 5, 2007

(54) Method and Apparatus for Bi-Axial Light Treatment

(76) Inventors: David Havell, Salt Lake City, UT (US); Aaron Best, Murray, UT (US)

Correspondence Address: KIRTON AND MCCONKIE, 60 EAST SOUTH TEMPLE,, SUITE 1800, SALT LAKE CITY, UT, 84111, US

(21) Appl. No.: 11/557,911

(22) Filed: Nov. 8, 2006

Related U.S. Application Data
(60) Provisional application No. 60/734,644, filed on Nov. 8, 2005.

(51) Int. Cl. A61N 5/06

(52) U.S. Cl. 607/88

(57) ABSTRACT

A method and apparatus for providing light therapy using a biaxial flexible circuit substrate with infrared light-emitting diodes dispersed throughout the substrate, which is housed within a housing unit and has a transparent cover, a reflective layer and a means for connecting to a power source, is disclosed.

### RELATED APPLICATIONS

This application claims priority to U.S. provisional patent application No. 60/734,644, filed Nov. 8, 2005, entitled Bi-Axial light Treatment Apparatus.

### BACKGROUND

1. Field of the Invention

The present invention relates to light therapy and more particularly, a method and system that utilize light therapy to treat and alleviate symptoms associated with the types of diseases or ailments for which light therapy offers relief.

2. Background of the Invention and Related Art

Symptoms of various ailments and maladies are often alleviated or eliminated by a treatment therapy known as “light therapy.” Ongoing research shows “light therapy” provides powerful therapeutic benefits. In fact, blood vessel constriction, which leads to neuropathy, is treated using light therapy, because studies indicate that the light itself, generated at specific wavelengths, increases the blood flow in a blood-constricted area of a patient. Consequently, increased blood flow allows nerve endings to re-grow and such re-growth consequently restores feeling where circulation is poor. Thus, light therapy offers the ability of alleviating and/or eliminating symptoms associated with the type of ailments for which light therapy responds.

Possible light sources for light therapy include light-emitting diodes (LEDs). Specifically, studies show using infrared LEDs effect changes, positively, at a cellular level, and that LEDs are effective light sources because they disperse light and heat over a great surface area of a patient without the risk of injury to soft tissue.

Among the many positive effects of light therapy, include: reversal of peripheral neuropathy; wound healing; reduction of pain and normal nerve conduction restoration; increased vascularity (circulation); reduction of edema (i.e., research shows that lymph vessel diameter and the flow of lymph system can be doubled with the use of light therapy); stimulation of collagen production; bone healing; and alleviation of symptoms associated with diabetes.1
¹“MaMMA Light Therapy in Relation to Diabetes Wound Healing” This article by: Sheila Khanna, M D, Angela Mullett-Silva, D C, Kathleen Linaker, D C, Quoc Giang, D C; http://72.14.253.104/search?q=cache:PPNvlzh7nYkJ:www.womensweb.ca/health/diabetes/diabetes2.php+light+therapy+diabetes&hl=en&gl=us&ct=clnk&cd=1

Current systems available for light therapy have drawbacks. Specifically, current light therapy techniques feature rigid structures, which are not capable of being conformed to and applied to various parts of the body. In other words, many systems lack the versatility of applying the system to different parts of the body. Moreover, some systems lose too much infrared energy during the transfer of light to the affected area of a light-therapy user's body because the systems inefficiently reflect light back to the body that has been reflected from the body. That is, any energy from the infrared light that is reflected by the body, is rarely, with current systems, re-directed back to the body. Finally, available systems are expensive and difficult to manufacture and/or mass produce.

A need, therefore, exists for a light therapy method and apparatus that is able to conform to a user's (or patient's) body, which can bend along two-axes and that efficiently, directs infrared energy or light into a user's body, and finally, is capable of inexpensive mass fabrication.

### SUMMARY AND OBJECTS OF THE INVENTION

An exemplary embodiment of the present invention provides a method and apparatus for providing light therapy to a user. Specifically, an exemplary embodiment of the present invention comprises a bi-axially bending flexible circuit substrate, housed within a flexible housing unit, which is capable of applying therapeutic light to a user's body. The apparatus and method of the exemplary embodiment can be applied to different parts of the body as it is bi-axially flexible and able to conform to the affected body part. Bi-axially flexible means the exemplary embodiment is able to bend in two-axes.

Moreover, in the exemplary embodiment, the light is effectively dispersed on the user's body by infrared LEDs located on the flexible circuit substrate that transfer through a reflective panel, and which continually reflects and re-directs the LEDs' light back to the user's body if the user's body reflects the light. This allows for optimal light transfer and therapy.

In the exemplary embodiment, the reflective panel that is on top of the flexible circuit substrate is further covered by a transparent cover. The transparent cover serves to protect the flexible circuit panel and the reflective panel from moisture. It also protects the user's skin from the LEDs intensity.

Moreover, the exemplary embodiment comprises a hook and loop means for securing the system (i.e., the apparatus and method for biaxial light treatment) to the user's body. Consequently, the system if flexible, versatile and easy to apply to needed areas of the body requiring light therapy. Moreover, the components of the system, and specifically, the flexible circuit substrate, utilize surface mounting technology, which renders the system capable of mass fabrication and production.

Accordingly, it is an object of the present invention to provide a method and apparatus for providing light therapy that can biaxially and flexibly conform to a user's (or patient's) body. That is, an object of the present invention enables a system for providing light therapy that can bend along two-axes and delivers therapeutic benefits of light while minimizing discomfort to the user.

Another object of the present invention provides a system having a reflective layer between the LEDs of the flexible circuit substrate and the skin of the user, which efficiently, directs infrared energy or light into a user's body, when in traditional light therapy systems, such energy is commonly lost.

Yet another object of the present invention is to provide a system for offering light therapy that is capable of inexpensive mass fabrication. Specifically, another object of the present invention is to employ surface mount technology that allows for high-volume manufacturing while also providing a flexible circuit substrate.

A further object of the present invention is to provide a system for offering light therapy to a user's body that can be hooked or fastened and conformed to any part of the user's body, regardless of the shape of the specific body part to which the system is being applied.

An even further object of the present invention is to provide a system for providing light therapy that is powered via battery or AC/DC power converters.

These and other objects of the present invention will become more fully apparent from the following description, drawings, and claims. Other objects will likewise become apparent from the practice of the invention as set forth hereafter.

### BRIEF DESCRIPTION OF THE DRAWINGS

The foregoing and other objects and features of the present invention will become more fully apparent from the accompanying drawings when considered in conjunction with the following description and appended claims. Although the drawings depict only typical embodiments of the invention and are thus, not to be deemed limiting of the invention's scope, the accompanying drawings help explain the invention in added detail.

FIG. 1 depicts an exploded view of an exemplary embodiment of the present invention. Specifically, FIG. 1(a) shows a power supply apparatus that provides power to the exemplary embodiment of the present invention shown in FIG. 1(b).

FIG. 2 depicts an exemplary embodiment of the present invention with the elements of this embodiment separated and unconnected from one another and in the sequence and configuration they would be found if connected.

FIG. 3 depicts a housing element. Specifically, FIG. 3 depicts a base pad and a top portion, which when connected to the base pad, forms the housing unit; the top portion being the top of the housing unit and the base pad comprising the bottom. FIG. 3 also shows how when the top portion connects with the base pad and encompasses the flexible circuit substrate between the top portion and the base pad, the housing unit is formed.

FIG. 4 depicts a hook and loop securing means of the exemplary embodiment, which enables the embodiment of the present invention to be attached to a user. Specifically, FIG. 4(a) depicts the loop fastener of the hook and loop securing means, while FIG. 4(b) depicts the hook pad.

FIG. 5 depicts a view of the hook pad of the hook and loop securing means before it is attached to the base pad of the housing unit.

FIG. 6 shows a reflective panel attaching to the skin-contacting side of the flexible circuit substrate.

FIG. 7 shows a transparent cover in a position above, and about to be received by, the reflective panel. When the system of the present invention is assembled, the reflective panel receives the transparent cover on top of it.

FIG. 8 depicts various perspectives of a power and switch box of the exemplary embodiment of the present invention. Specifically, FIG. 8(a) shows the power and switch box having all elements separated and unconnected from one another. FIG. 8(b) depicts the power and switch box with only the lid of the power and switch box removed and in a position above the power and switch box housing area. FIG. 8(c) shows the power and switch box in its assembled state, as it would appear if about to be operated. Finally, FIG. 8(d) shows an AC/DC transformer that enables power to transfer to the power and switch box, and ultimately, to the flexible circuit substrate.

FIG. 9 depicts perspectives of the flexible circuit substrate. Specifically, FIG. 9(a) is a bird's eye view of the flexible circuit substrate, FIG. 9(b) is a side view, and FIG. 9(c) is a close-up of the side view of the flexible circuit substrate and the Light-Emitting Diode's.

FIG. 10(a) is a detailed layout of the control board of the power and switch box. FIG. 10(b) is another depiction of the layout of the control board.

FIG. 11(a) and 11(b) are schematics of the diode arrays of the flexible circuit substrate.

### DETAILED DESCRIPTION OF THE PREFERRED EMBODIMENTS

It is emphasized that the present invention, as illustrated in the figures and description herein, can be embodied in other forms. Thus, neither the drawings nor the following more detailed description of the various embodiments of the system and method of the present invention limit the scope of the invention. The drawings and detailed description are merely representative of examples of embodiments of the invention; the substantive scope of the present invention is limited only by the appended claims recited to describe the many embodiments. The various embodiments of the invention will best be understood by reference to the drawings, wherein like elements are designated by like alphanumeric character throughout.

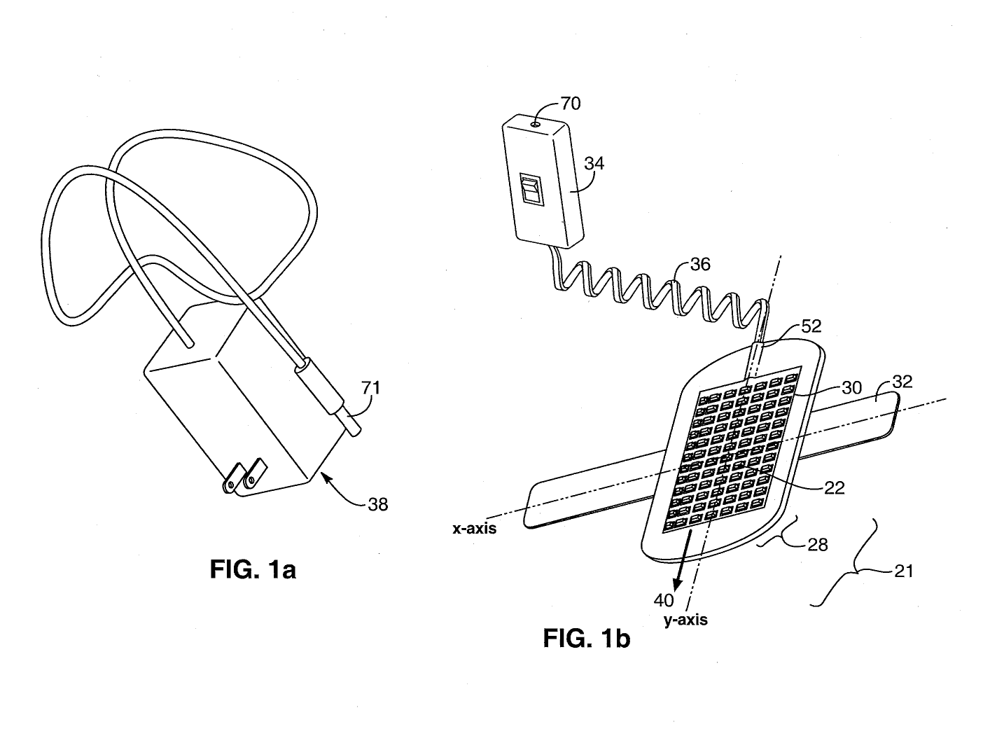

With reference now to the accompanying drawing, FIG. 1 shows an exemplary embodiment of the present invention. Specifically, FIG. 1(a) shows a power supply apparatus 38 that provides power to the exemplary embodiment of the present invention shown in FIG. 1(b).

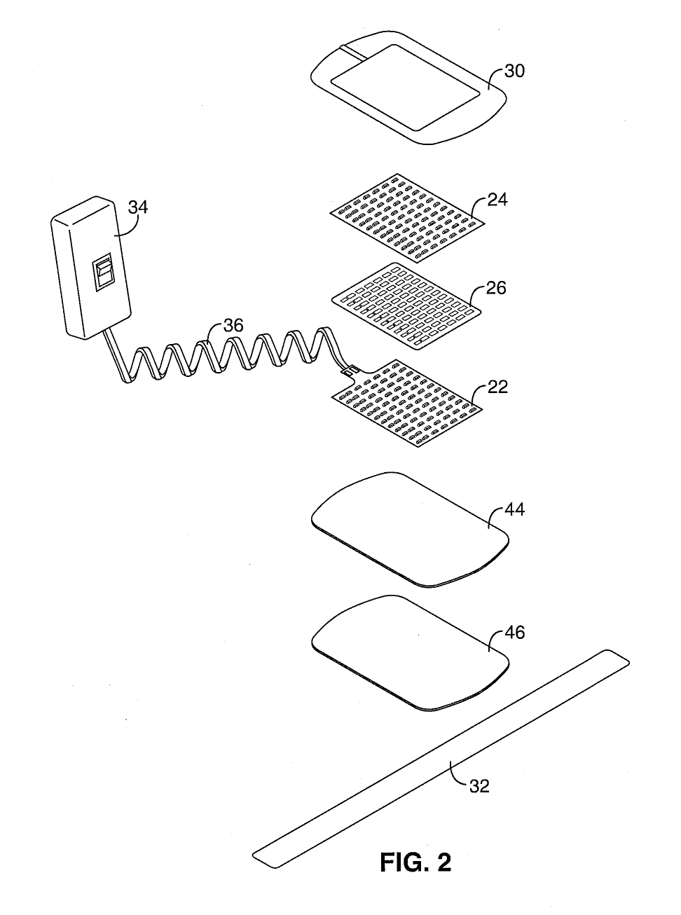

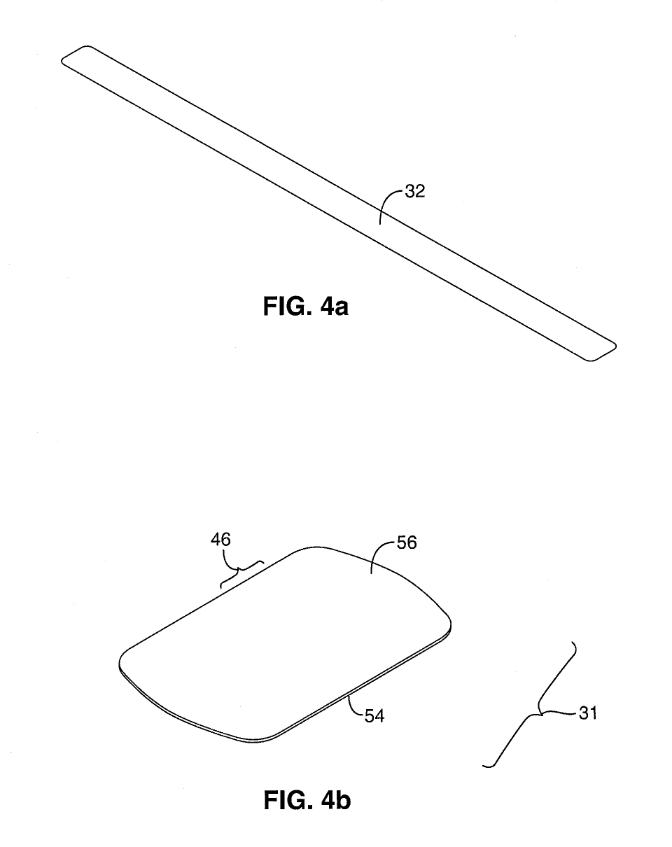

FIG. 1(b) shows a flexible circuit substrate 22 (with a transparent cover 24 and a reflective panel 26 over the substrate 22, though not readily apparent in this figure, but illustrated in detail in FIGS. 2, 6 and 7), as it is housed within a housing unit 28. The top portion 30 of the housing unit 28 is also illustrated. Moreover, the loop fastener 32 of the hook and loop means (also not shown in this FIG. 1(b), but illustrated in FIGS. 2 and 4). FIG. 1(b) also depicts a power and switch box 34 as well as a cord 36 that attaches from the flexible circuit substrate 22 to the power and switch box 34. A power supply apparatus 38 (such as an AC/DC transformer) provides power to the flexible circuit substrate 22, which ultimately, activates LEDs on the flexible circuit substrate 22. One end 71 of the power supply element 38 attaches to holes 70 of the power and switch box 34. The power supply apparatus 38 is shown in FIG. 1(a).

In greater detail, the exemplary embodiment of the invention displays a system 21 that uses infra-red treatment on a user for which such treatment works. Specifically, the system 21 provides an array of high-intensity surface mounted infra-red LEDs 40 on a bi-axially rotational and flexible circuit substrate 22. Alternatively, the system need not use infra-red LEDs 40 as its light source, but may be comprised of any light sources, i.e., light bulbs, lasers or other light sources generally acceptable to medical personnel for delivering light and heat. The light source 40 may be placed in a flared light pattern. However, the exemplary embodiment utilizes surface mounted LEDs 40, which allow manufacturers to print the flexible circuit substrate 22 with receiver sockets in which the LEDs 40 can be placed. The surface mounting technique for LEDs 40 placement enables the user to replace LEDs as they begin to fail, thus lowering replacement and maintenance costs associated with replacing the entire apparatus 21. In addition, surface mounted LEDs 40 allow for high volume manufacturing and are individually replaceable. The LEDs 40 may be arranged in a general grid-like pattern or in patterns specifically adapted to specific anatomical structures.

This system 21 is applied to a user's skin and provides light therapy for ailments suffered by the user, for which light therapy alleviates symptoms. The flexible circuit substrate 22 is bendable around both an x and y-axis shown in FIG. 1 so that it may easily conform to the user's parts of the body requiring light therapy. Thus, the system delivers therapeutic benefits of light while minimizing discomfort to the user. The system 21 works by increasing blood flow in the immediate area to which the system is applied and may be used to treat neuropathy, among other maladies caused by blood vessel constriction. The increase of blood flow allows nerve endings to re-grow and restores feeling in the extremities of neuropathy patients.

The system has a reflective panel 26 (not readily shown in FIG. 1, but in figures viewable in 2 and 6) that adheres or is attached to the flexible circuit substrate 22 and encompasses the LEDs 40, which directs all light emitted from the LEDs 40 onto the surface of the epidermis of the user. Again, the system 21 is flexible and conforms readily to any curved surface to which it is applied.

FIG. 2 depicts an exemplary embodiment with the elements of this embodiment separated and unconnected from one another. Specifically, FIG. 2 shows a possible order for which the elements are placed together. In other words, FIG. 2 shows the elements in sequence, as if the system 21 were disassembled and about to be re-assembled. The flexible circuit substrate 22 connects to the power and switch box 34 via a cord 36, and is the central element of the system 21. Immediately above the flexible circuit substrate 22, the reflective panel 26 is shown. Immediately above the reflective panel 26, the transparent cover 24 is shown. The top portion 30 of the housing unit 28 is shown above the transparent cover 24. Moreover, the top portion 30 of the housing unit 28, when integrally connected to the base pad 44 of the housing unit 28 (which is shown immediately beneath the flexible circuit substrate 22), forms the housing 28 that encompasses the system. The housing unit 28 is not shown in this figure because the top portion 30 and the base pad 44 that comprise the housing unit 28, are not connected.

In order to secure the system to the user, a hook and loop securing means is used. The loop fastener 32 of the hook and loop securing means 31 (not shown) is shown at the bottom of FIG. 2, and is located immediately beneath the hook pad 46. By the hook and loop securing means 31, the system 21 has the ability to attach to different parts of the body. The system can be used as a single system or in a set of multiple systems. The housing unit 28 can be manufactured into different shapes to cover the anatomical part needing treatment, i.e., to cover the foot, hand, face, back, etc.

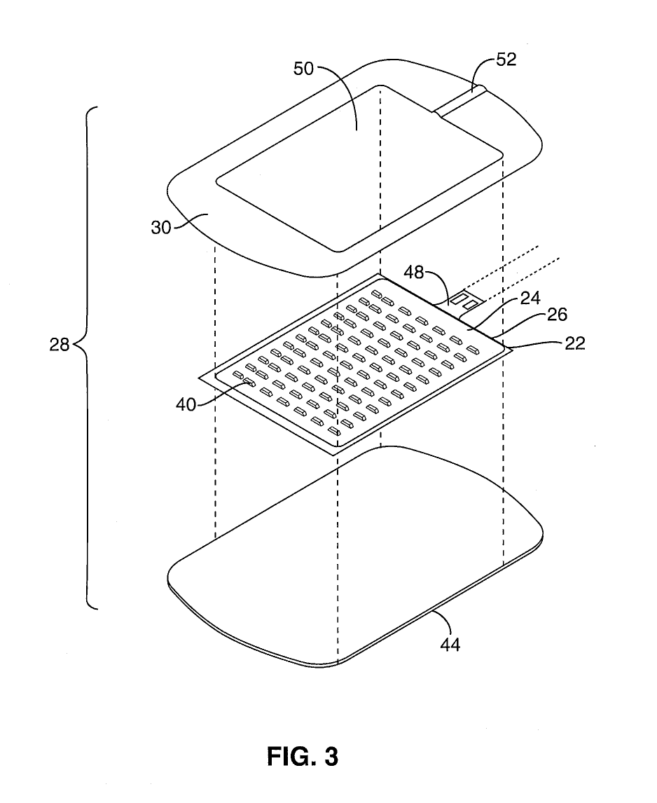

FIG. 3 depicts the housing element 28 as it is about to encompass the flexible circuit substrate. Specifically, FIG. 3 depicts a base pad 44 and a top portion 30 that when connected to the base pad 44, forms the housing unit 28; the top portion 30 being the upper-most part of the housing unit 28 and the base pad 44 comprising the bottom-region of the housing unit. The top portion 30 may be composed of neoprene or material with similar properties. The base pad 44 may also be composed neoprene, or materials and compositions with similar properties. In the exemplary embodiment, the top portion 30 and the base pad 44 are constructed of 0.060″ of neoprene rubber. The top portion 30 and the base pad 44 provide the strength of the system 21 and the housing unit 28. Using neoprene provides the flexibility and comfort the user experiences when the system contacts his or her skin.

The housing unit 28 encompasses within it, the system 21 for emitting heat therapy, and specifically, encompasses the flexible circuit substrate 22, reflective panel 26 and transparent cover 24, when the foregoing elements are joined together. The base pad 44 is solid, while the top portion 30 has an opening 50 circumscribing the system, which is “sandwiched” within. By providing the opening 50, light and heat may be received on the skin of the user, however, at the areas of the top portion 30 where there is no opening 50, light and heat are prevented from escaping. This allows the user to use the system 21 where he or she desires, without having undesired areas of the skin affected. Additionally, the top portion 30 may be selectively releasable from the base pad 44 to allow the user to conveniently replace parts of the system as desired.

A method of securing the top portion 30 and the base pad 44 would be to have them sewn or similarly attached around the edges. It might also be necessary to apply a small amount of epoxy (3M DP190) to the cord 36 (not shown) and either the top portion 30 of the housing unit or the base pad 44 or both as strain relief. The housing unit tunnel 52 receives the cord 36 from the power and switch box 34 so that it can attach to the connection element 48 of the flexible circuit substrate 22. The connection of the connection element and the cord 36 enables power from the power and switch box to be received by the flexible circuit substrate 22.

FIG. 4 depicts a hook and loop securing means 31 of the exemplary embodiment that enables the embodiment of the present invention to be attached to a user. Specifically, FIG. 4(a) depicts the loop fastener 32 of the hook and loop securing means 31, while FIG. 4(b) depicts the hook pad 46. The hook and loop securing means 31 is adjustable to accommodate the different areas of the body to which the system 21 is attached and to maximize comfort for the user. The hook pad 46 is the area to which the loop fastener 32 is “hooked” or attached. That is, the loop fastener 32 may be attached to the hook pad 46 because both are made of a composition, such as VELCRO® that connects one area of an element to another area of a different element. In this context, the loop fastener 32 attaches to the hook pad 46 and consequently, to the system 21, and also attaches to the user. Thus, the hook and loop securing means 31 may comprise material where one side of the material is a hook and the opposite side of the material is loop or pile. The hook and loop securing means 31 may comprise double-sided hook and loop securing means 31. The loop fastener 32 may be double-sided VELCRO® so that it connects to itself as it is wrapped around the area requiring treatment of the user. The loop fastener 32 may be composed of any material that allows connection of the element to itself, VELCRO® is merely an example. The hook pad 46 is also composed of VELCRO® in the exemplary embodiment so that it may attach to any portion of the loop fastener 32. The securing means 31 ultimately enables the system 21 to be fastened to any part of the body in any orientation.

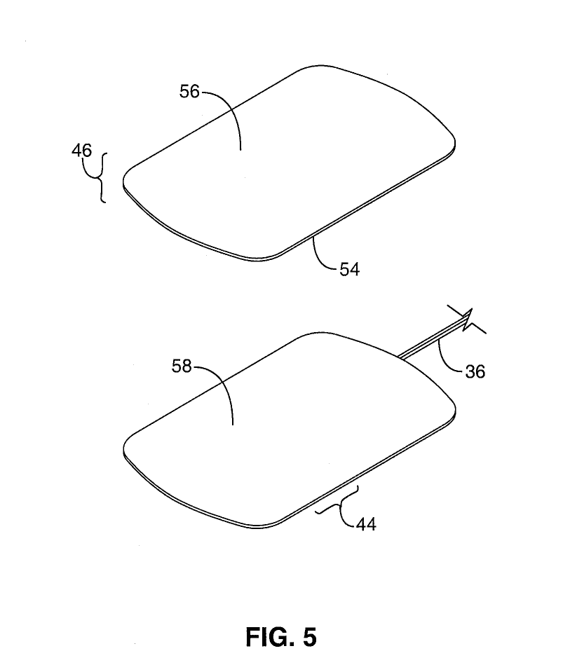

FIG. 5 depicts the hook pad 46 of the hook and loop securing means 31 before it is attached to the base pad 44 of the housing unit 28. In this figure, the base pad 44 is in an upside down position, that is, the flexible circuit substrate 22 is not viewable as it is face down. The hook pad 46 is attached to the bottom 58 of the base pad 44 and is attached to by either glue or similar means of attaching the two elements. Not shown in this Figure is the loop fastener 32, which attaches to one side 56 of the hook pad 46. The latter connection is illustrated in FIG. 4.

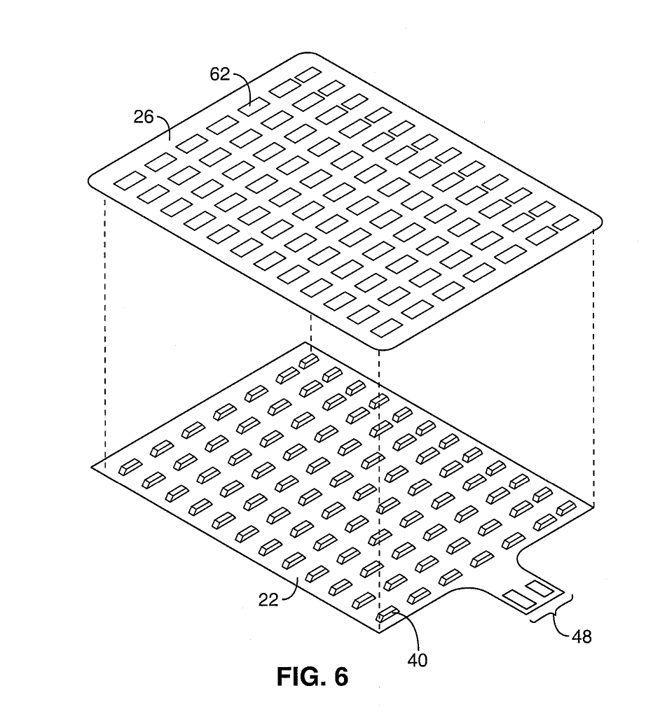

FIG. 6 depicts the reflective panel 26 as it is about to be attached to the flexible circuit substrate 22, and specifically, as it attaches to the area of the flexible circuit substrate 22 that faces the area of skin to be treated. In order to maximize the amount of light and heat reflected onto the user's skin, the reflective panel 26 is placed on top of the flexible circuit substrate 22 and has a series of ports 62 capable of receiving the light bulbs, LEDs 40 or other light sources of the flexible circuit substrate 22. The reflective panel 26 directs any light and heat not oriented toward the skin, back to the skin so that the amount of heat and light exposed to the skin is maximized. The reflective panel 26 is made out of metalized polymer, such as vinyl or Mylar and similarly composed properties. This reflective panel 26 may be adhered to the flexible circuit substrate 22 or integrally connected to it at the time the flexible circuit substrate's manufacture. It 26 may have adhesive on it 26 to adhere to the flexible circuit substrate 22. In other words, the reflective panel 26 may be built as part of the flexible circuit substrate 22. Again, the purpose of the reflective panel 26 is to reflect light back toward the user's skin that would be otherwise absorbed into the base pad 44 of the housing unit 28. The reflective panel 26, similarly to the flexible circuit substrate 22, is biaxially flexible. The reflective panel 26 increases the efficiency of the system 21 and decreases the power needed for treatment. An example of the reflective panel 26 is Mylar film.

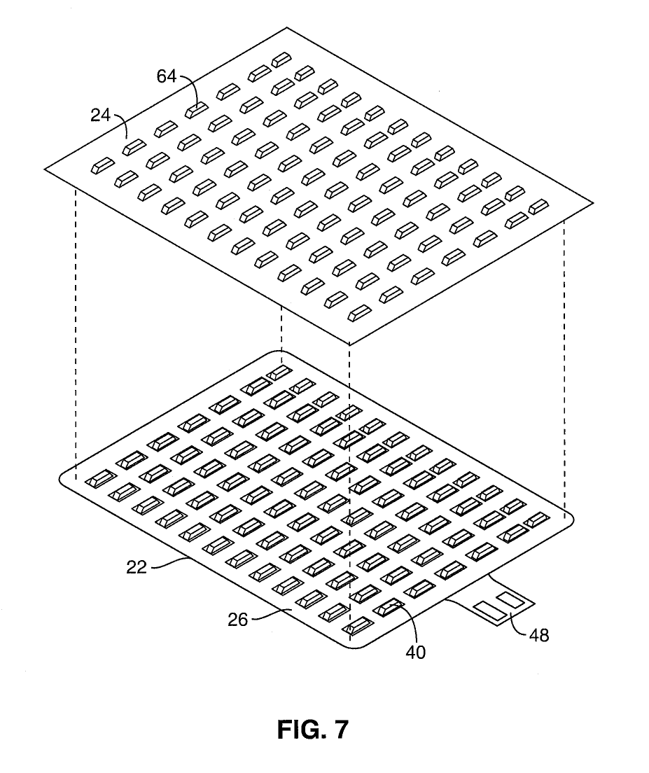

FIG. 7 is a transparent cover 24 and is shown above the reflective panel 26 as it is attached to the flexible circuit substrate 22. The reflective panel 26 receives the transparent cover 24 on top of it. The transparent cover 25 is like a translucent or transparent film that when placed on top of the reflective panel 26, protects and covers the reflective panel 26. In other words, the transparent cover's 24 purpose is to keep the elements of the system from having direct contact with the skin. It also helps to seal the system 21 from unwanted moisture and protects the circuits. The transparent cover 24 also increases the comfort of the system 21 when worn against the skin. The transparent cover 24 is replaceable so that if the transparent cover 24 becomes dirty or destroyed, it can be replaced without damage to the reflective panel 26 or the flexible circuit substrate 22. The transparent cover 24 can be vinyl and is either thermoformed directly onto the reflective panel 26/flexible circuit substrate 22 assembly or molded by injection mold or vacuum formed to the cover 24 separately and then attached to the reflective panel 26 with adhesive. FIG. 7 also shows a connection element 48 that connects the cord 36 (not shown) to the power and switch box 34 (also not shown in this Figure, but viewable in FIG. 2.)

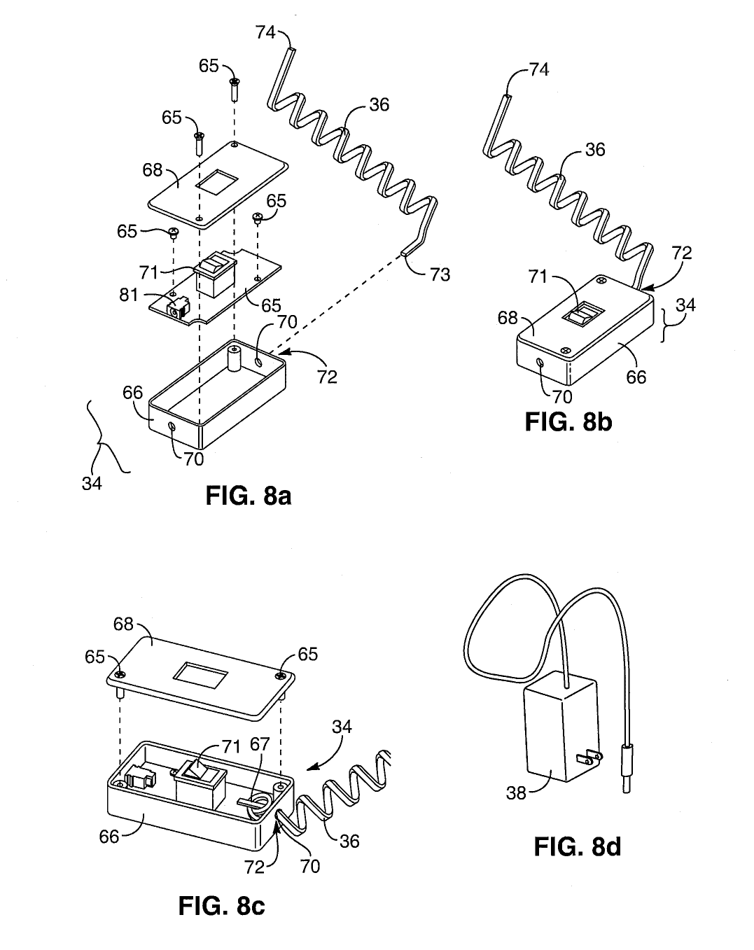

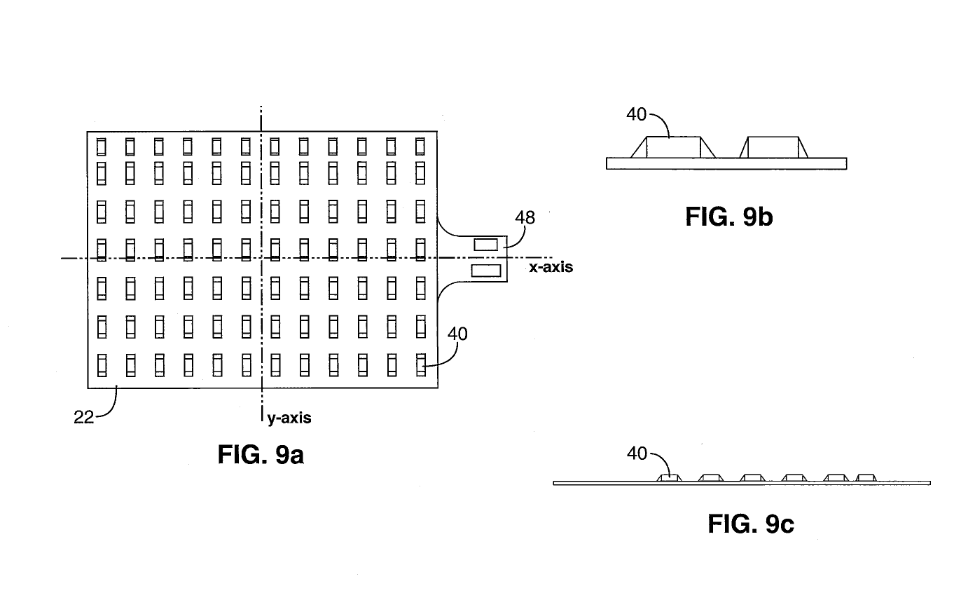

FIG. 8 depicts various perspectives of a power and switch box of the exemplary embodiment of the present invention. Specifically, FIG. 8(a) shows the power and switch box 34 with all of its elements separated and unconnected from one another. FIG. 8(b) depicts the power and switch box 34 with the lid 68 and the power control board 69 of the power and switch box 34 removed and in a position above the power and switch box housing 72 area. The power and switch box housing area 72 has diametrically opposed holes 70. One end is for the power supply 38 (i.e, AC/DC transformer, battery power source, etc.) while the other is for receiving the cord 36 that runs from the power and switch box 34 to the flexible circuit substrate 22. The lid 68, the power control board 69 and the power and switch board housing unit 72 may be secured by screws 65 or similar elements used for holding items together. FIG. 8(c) shows the power and switch box 34 in its assembled state, as it would appear if about to be operated. When the switch 71 is turned on and power is applied, an LED (not shown) will light on the body of the switch 71. The power and switch box 34 has a voltage regulator built into its circuitry that allows the system 21 to operate on any voltage from nine to 23 volts DC. The multi-volt capability of this system 21 means it can operate using both the wall mount transformer 38, power from a standard automobile outlet, or batteries. FIG. 8(d) shows only the lid 68 of the power and switch box 34 unattached from the power and switch box 34. This figure illustrates that the cord 36 is inserted through one of the diametric end holes 70. Enough of the cord 36 is left dangling within the power and switch box housing area 72 and within one of the diametric end's holes 70 that a knot 67 can be tied to provide strain relief. The cord 36 may then be soldered or connected to the board at the connection end 48 (not shown) of the flexible circuit substrate 22 (not shown) via the housing unit tunnel 52. The lid 68 can then be secured with screws 65. Finally, FIG. 9(e) shows an AC/DC transformer 38 that enables power to run to the power and switch box 34, and ultimately, to the flexible circuit substrate 22 so that the LEDs 40 of the flexible circuit substrate 22 emit light and heat. The power supply apparatus 38 may be either a standard 110 or 220 volt system or a battery powered system. If the system is used in mobile situations, it is anticipated that the power source is a battery. The section of the cord 36 that connects to the power and switch box housing unit is the cord's first end 73 and the section of the cord 36 that connects to the flexible circuit substrate's 22 connection element 48 via the housing unit tunnel 52 is the cord's second end 74.

FIG. 9 depicts various perspectives of the flexible circuit substrate 22. Specifically, the flexible circuit substrate 22 is shown from a bird's eye view (FIG. 9(a)); a side view with the LEDs enlarged and close-up (9(b)); and another side view (9(c)). The flexible circuit substrate may be KAPTON®, also known as the polyimid flexible circuit, with LEDs. Again, the flexible circuit substrate 22 is flexible around two axes, the x and y-axis. Using flexible circuit substrates 22 with LEDs 40 are optimal because they are capable of mass production and of using surface mount technology.

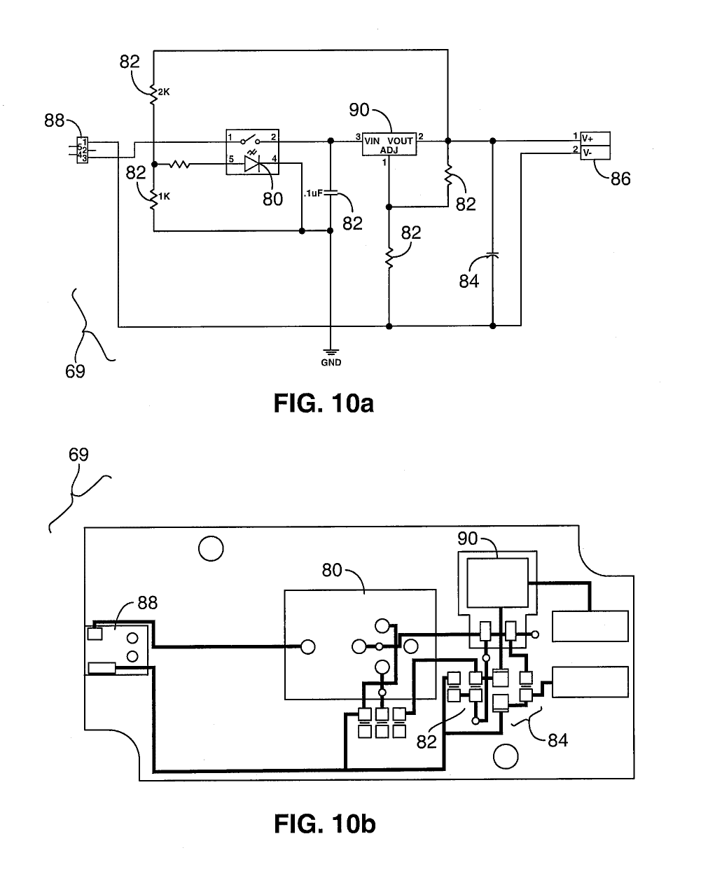

FIG. 10(a) is a detailed layout of the power control board 69 of the power and switch box 34 that enables the power and switch box 34 to operate. The design of the power control board 69 is done using standard electronic components. DC power from 9V to 23V is applied to the power connector 88. When the switch 71 is on, the Voltage Regulator 90 outputs the correct voltage and supplies enough current to energize the diode array through the Solder Pads 86. The capacitors 84 are for stabilization and improved transient response. The resistors 82 set the proper voltage and current for both the Voltage Regulator 90 and the LED 40 inside the switch 71. The board 69 may be standard FR4 material and 0.060″ thick and manufactured at any typical PCB facility throughout the world. FIG. 10(b) is another depiction of the layout of the power control board 69. Again, all components are surface mountable and pick-and-place-able. The solder pads 86 are HASL-finished solderable pads 86 where the cord 36 (via end 73) can attach to the flexible circuit substrate's 22 connection end 48 (not shown) via the housing unit tunnel 52.

An embodiment may also generate a magnetic field or flux across the skin. An additional flexible circuit may be added behind the light array and may produce a magnet field. The magnetic flux would also be directed toward the skin. The magnetic flux may be generated by passing a current through a wire, utilizing hard magnets in the apparatus, or any other means known in the art. The magnitude of the magnetic fields may be adjusted by modifying the amount of current passing through the wire or by changing the distance or side of the hard magnets used.

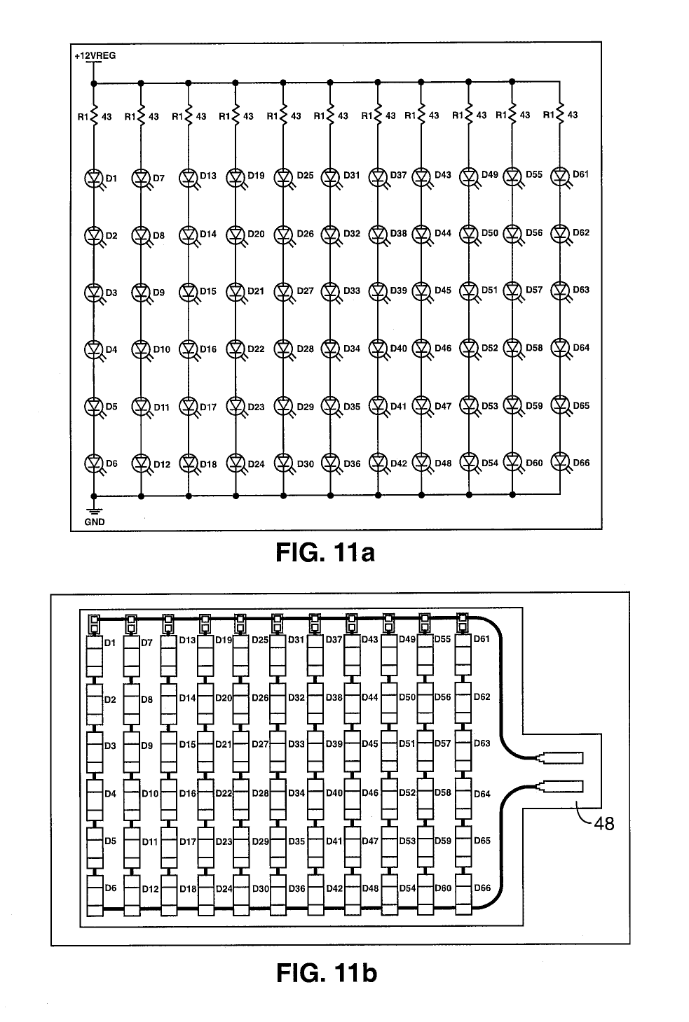

FIGS. 11(a) and 11(b) are more schematics of diode arrays. Regulated voltage powers each series branch of parallel branches. Diodes are arranged in series to ensure each diode experiences the same current. Resistors set the current through each branch. Each series branch is paralleled together to minimize the amount of current dissipated in the current setting resistors. Resistor values and the regulated voltage applied can be carried to achieve optimum power output of diodes and minimal amount heat generated in the resistors. In FIGS. 11(a) and 11(b), and the exemplary embodiment, there are 66 LED's 40 that are surface mounted infrared diodes. The diodes are spaced closely and evenly across the board to achieve high power density.

What is claimed:

1. An apparatus for providing light therapy to skin of a body, comprising:
a bi-axial flexible circuit substrate having at least one light source, wherein when the light source is activated by a power supply source, the light source provides a therapeutic amount of light to the skin of the body to which the flexible circuit substrate is attached.

2. The apparatus of claim 1 further comprising:
a plurality of light sources on the bi-axial substrate, wherein when the light sources are activated by a power supply source, the light sources provide a therapeutic amount of light to the skin of the body to which the flexible circuit substrate is attached.

3. An apparatus in claim 2, wherein the plurality of light source comprises light-emitting diodes.

4. An apparatus in claim 3, wherein the light-emitting diodes emit infrared energy.

5. An apparatus as in claim 3, wherein the light-emitting diodes are arranged in a grid-like pattern that optimally mimics a specific anatomical structure.

6. An apparatus as in claim 2, wherein the power source is selected from the group consisting of: AC/DC transformer, battery power source and car power source.

7. An apparatus as in claim 2, wherein the means for connecting the substrate to the body comprises a hook and loop securing means.

8. An apparatus as in claim 7, wherein the hook and loop securing means comprises double-sided VELCRO®.

9. An apparatus as in claim 2, wherein the plurality of light sources are surface mounted on top of the flexible circuit substrate.

10. An apparatus as in claim 2, further comprising a reflective panel that is located on top of the flexible circuit substrate.

11. An apparatus as in claim 2, wherein the reflective panel is integrally connected to the flexible circuit substrate.

12. An apparatus as in claim 11, wherein the reflective panel operates to re-direct energy or light emitted from the light source and reflected by the body, back into the body.

13. An apparatus as in claim 10, further comprising a transparent cover, wherein the transparent cover serves as a protection for the skin as well as for the flexible circuit substrate and covers the reflective panel and flexible circuit substrate, respectively.

14. An apparatus as in claim 10, wherein the transparent cover is made from vinyl.

15. An apparatus as in claim 10, wherein the transparent cover is thermoformed directly on the reflective panel and the flexible circuit substrate.

16. An apparatus as in claim 2 further comprising a second flexible circuit generating a magnetic field may be generated across the skin, wherein the magnitude of the magnetic fields may be selectively adjusted.

17. An apparatus for providing light therapy to skin of a body, comprising:
a bi-axial flexible circuit substrate having a plurality of light sources, wherein the light sources, when activated, provide a therapeutic amount of light to the skin of the body to which the flexible circuit substrate is attached;
a cord that connects the substrate to a power and switch box;
a power supply source that connects to the power and switch box and enables power to be received by the power and switch box as well as by the flexible circuit substrate; and
a means for connecting the substrate to the body.

18. An apparatus of claim 17, wherein the plurality of light sources is surface mounted on top of the flexible circuit substrate.

19. An apparatus of claim 17, further comprising a reflective panel that serves to re-directed energy or light produced from the light sources that are reflected from the skin, back onto the skin.

20. An apparatus of claim 17, further comprising a transparent cover that covers the reflective panel as the reflective panel covers the flexible circuit substrate.

21. An apparatus of claim 17, wherein a housing unit encompasses the transparent cover as it exists on top of the reflective panel, which is on top of the flexible circuit substrate, and fastens to a hook and securing means, which is comprised of a hook pad and a loop fastener.

22. An apparatus of claim 21, wherein the hook and loop securing means comprise double-side VELCRO®.

23. A system for delivering light therapy to skin on a body comprising:
a biaxial flexible circuit substrate with LEDs protruding from the substrate;
a power supply source connected via a power and switch box, which when a switch on the power and switch box is activated, the LEDs on the flexible circuit substrate emit light;
a hook and securing means attached to the flexible circuit substrate so that when the light from the LEDs may provide therapeutic energy onto the skin.

24. A system as described in claim 23, wherein the LEDs permeate infrared onto desired areas of the body.

25. A system as described in claim 23, wherein the LEDs are surface mounted onto the biaxial flexible substrate.

26. A method using an apparatus that delivers a therapeutic amount of light to skin of a body, the method comprising the steps of:
positioning on the body a biaxial flexible circuit substrate having at least one light source provided thereon;
enabling the light source to be directed onto the skin of a body by means of a hook and securing means; and
providing a reflective panel that optimizes the amount of light directed onto the skin.

27. A method as in claim 26, wherein the light source is surface mounted onto the flexible circuit substrate.
